(19) United States
(12) Patent Application Publication
(10) Pub. No.: US 2007/0191247 A1
(43) Pub. Date: Aug. 16, 2007

(54) Detergent compositions

(75) Inventors: Philip Frank Souter, Northumerland (GB); John Allen Burdis, Newcastle upon Tyne (GB); Neil Joseph Lant, Newcastle upon Tyne (GB)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/656,117

(22) Filed: Jan. 22, 2007

Related U.S. Application Data
(60) Provisional application No. 60/761,187, filed on Jan. 23, 2006.
(60) Provisional application No. 60/795,964, filed on Apr. 28, 2006.
(60) Provisional application No. 60/854,836, filed on Oct. 27, 2006.

(51) Int. Cl. C11D 3/386; C12N 9/18; C11D 3/28

(52) U.S. Cl. 510/320; 435/197

(57) ABSTRACT

This invention relates to compositions comprising certain lipase variants and a photobleach and processes for making and using such compositions. Including the use of such compositions to clean and/or treat a situs.

### CROSS-REFERENCES TO RELATED APPLICATIONS

This application claims priority under 35 U.S.C. §119(e) to U.S. Provisional Application Ser. No. 60/761,187 filed Jan. 23, 2006, U.S. Provisional Application Ser. No. 60/795,964 filed Apr. 28, 2006, and U.S. Provisional Application Ser. No. 60/854,836 filed Oct. 27, 2006.

### FIELD OF THE INVENTION

This invention relates to compositions comprising lipases and photobleaches and processes for making and using such products.

### BACKGROUND OF THE INVENTION

The appearance of lipase enzymes suitable for detergent applications gave the formulator a new approach to improve grease removal. Such enzymes catalyse the hydrolysis of triglycerides which form a major component of many commonly encountered fatty soils such as sebum, animal fats (e.g. lard, ghee, butter) and vegetable oils (e.g. olive oil, sunflower oil, peanut oil). However these enzymes typically showed weak performance in the first wash cycle and typically came with a malodor arising, it is believed, from hydrolysis of fats present in dairy soils like milks, cream, butter and yogurt. While not being bound by theory, it is believed that such soils are prone to lipase-induced malodor generation as they contain triglycerides functionalized with short chain (e.g. C₄) fatty acyl units which release malodorous volatile fatty acids after lipolysis. Even the when the performance of such enzymes was improved, the malodor issue remained. Thus, the use of this technology was severely limited.

We have found that the combination of a photobleach with certain lipase variants gives rise to an improved cleaning performance benefit, while minimising unacceptable malodor. Without wishing to be bound by theory, it is believed that the following mechanisms are likely to give rise to such benefits: improved stain removal of stains comprising carotenoid, anthocyanines, porphyrins, tannins and flavines materials, for example, curry, pepper sauce, tomato-based pasta sauces, coffee and tea, due to synergistic action between the lipase and photobleach; and the oxidation of the lipase enzyme, by the photobleach, post-wash, for example during the drying of the cleaned or treated situs thus leading to reduced malodor.

### SUMMARY OF THE INVENTION

The present invention relates to compositions comprising a photobleach and a lipase variant with reduced potential for odor generation and a good relative performance, without the attachment of a C-terminal extension. The lipase variant is obtained by introducing mutations in one or more regions identified in the parent lipase. The variant thus obtained must have a lipase activity which is not less than 80% of the parent lipase's activity expressed as Relative Performance.

### BRIEF DESCRIPTION OF THE FIGURES

FIG. 1 shows the alignment of lipases.

### SEQUENCE LISTINGS

SEQ ID NO: 1 shows the DNA sequence encoding lipase from Thermomyces lanoginosus.

SEQ ID NO: 2 shows the amino acid sequence of a lipase from Thermomyces lanoginosus.

SEQ ID NO: 3 shows the amino acid sequence of a lipase from Absidia reflexa.

SEQ ID NO: 4 shows the amino acid sequence of a lipase from Absidia corymbifera.

SEQ ID NO: 5 shows the amino acid sequence of a lipase from Rhizomucor miehei.

SEQ ID NO: 6 shows the amino acid sequence of a lipase from Rhizopus oryzae.

SEQ ID NO: 7 shows the amino acid sequence of a lipase from Aspergillus niger.

SEQ ID NO: 8 shows the amino acid sequence of a lipase from Aspergillus tubingensis.

SEQ ID NO: 9 shows the amino acid sequence of a lipase from Fusarium oxysporrum.

SEQ ID NO: 10 shows the amino acid sequence of a lipase from Fusarium heterosporum.

SEQ ID NO: 11 shows the amino acid sequence of a lipase from Aspergillus oryzae.

SEQ ID NO: 12 shows the amino acid sequence of a lipase from Penicillium camemberti.

SEQ ID NO: 13 shows the amino acid sequence of a lipase from Aspergillus foetidus.

SEQ ID NO: 14 shows the amino acid sequence of a lipase from Aspergillus niger.

SEQ ID NO: 15 shows the amino acid sequence of a lipase from Aspergillus oryzae.

SEQ ID NO: 16 shows the amino acid sequence of a lipase from Landerina penisapora.

### DETAILED DESCRIPTION OF THE INVENTION

### DEFINITIONS

As used herein, the term “cleaning composition” includes, unless otherwise indicated, granular or powder-form all-purpose or “heavy-duty” washing agents, especially laundry detergents; liquid, gel or paste-form all-purpose washing agents, especially the so-called heavy-duty liquid types; liquid fine-fabric detergents; hand dishwashing agents or light duty dishwashing agents, especially those of the high-foaming type; machine dishwashing agents, including the various tablet, granular, liquid and rinse-aid types for household and institutional use; liquid cleaning and disinfecting agents, including antibacterial hand-wash types, laundry bars, mouthwashes, denture cleaners, car or carpet shampoos, bathroom cleaners; hair shampoos and hair-rinses; shower gels and foam baths and metal cleaners; as well as cleaning auxiliaries such as bleach additives and “stain-stick” or pre-treat types.

As used herein, the phrase “is independently selected from the group consisting of . . . ” means that moieties or elements that are selected from the referenced Markush group can be the same, can be different or any mixture of elements.

The test methods disclosed in the Test Methods Section of the present application must be used to determine the respective values of the parameters of Applicants' inventions.

Unless otherwise noted, all component or composition levels are in reference to the active level of that component or composition, and are exclusive of impurities, for example, residual solvents or by-products, which may be present in commercially available sources.

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All documents cited are, in relevant part, incorporated herein by reference; the citation of any document is not to be construed as an admission that it is prior art with respect to the present invention.

### Compositions

The compositions of the present invention typically contain from about 0.0001% to about 1%, from about 0.0002% to about 0.5%, or even from about 0.0005% to about 0.3% photobleach and from about 0.0005% to about 0.1%, from about 0.001% to about 0.05%, or even from about 0.002% to about 0.03% lipase.

Such compositions may take any form, for example, the form of a cleaning composition and/or a treatment composition.

The balance of any aspects of the aforementioned cleaning compositions is made up of one or more adjunct materials.

### Suitable Lipase Variants

The lipase of the composition of the present invention is a lipase variants with no C-terminal extension but with mutations introduced in certain regions of a parent lipase whereby the tendency to odor generation is reduced.

### Parent Lipase

The parent lipase may be a fungal lipase with an amino acid sequence having at least 50% homology as defined in the section “Homology and aligment” to the sequence of the T. lanuginosus lipase shown in SEQ ID NO: 2.

The parent lipase may be a yeast polypeptide such as a Candida, Kluyveromyces, Pichia, Saccharomyces, Schizosaccharomyces, or Yarrowia polypeptide; or more preferably a filamentous fungal polypeptide such as an Acremonium, Aspergillus, Aureobasidium, Cryptococcus, Filobasidium, Fusarium, Humicola, Magnaporthe, Mucor, Myceliophthora, Neocallimastix, Neurospora, Paecilomyces, Penicillium, Piromyces, Schizophyllum, Talaromyces, Thermoascus, Thielavia, Tolypocladium, or Trichoderma polypeptide.

In a preferred aspect, the parent lipase is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis polypeptide having lipase activity.

In another preferred aspect, the parent lipase is an Aspergillus aculeatus, Aspergillus awamori, Aspergillus fumigatus, Aspergillus foetidus, Aspergillus japonicus, Aspergillus nidulans, Aspergillus niger, Aspergillus oryzae, Aspergillus turbigensis, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Thermomyces lanoginosus (synonym: Humicola lanuginose), Mucor miehei, Myceliophthora thermophila, Neurospora crassa, Penicillium purpurogenum, Trichoderma harzianum, Trichoderma koningii, Trichoderma longibrachiatum, Trichoderma reesei, or Trichoderma viride polypeptide.

In another preferred aspect, the parent lipase is a Thermomyces lipase.

In a more preferred aspect, the parent lipase is a Thermomyces lanuginosus lipase. In an even more preferred embodiment the parent lipase is the lipase of SEQ ID NO: 2.

### Identification of Regions and Substitutions.

The positions referred to in Region I through Region IV below are the positions of the amino acid residues in SEQ ID NO:2. To find the corresponding (or homologous) positions in a different lipase, the procedure described in “Homology and alignment” is used.

### Substitutions in Region I

Region I consists of amino acid residues surrounding the N-terminal residue E1. In this region it is preferred to substitute an amino acid of the parent lipase with a more positive amino acid. Amino acid residues corresponding to the following positions are comprised by Region I: 1 to 11 and 223-239. The following positions are of particular interest: 1, 2, 4, 8, 11, 223, 227, 229, 231, 233, 234 and 236. In particular the following substitutions have been identified: X1N/*, X4V, X227G, X231R and X233R.

In a preferred embodiment the parent lipase has at least 80%, such as 85% or 90%, such as at least 95% or 96% or 97% or 98% or 99%, identity to SEQ ID NO:2. In a most preferred embodiment the parent lipase is identical to SEQ ID NO: 2.

### Substitutions in Region II

Region II consists of amino acid residues in contact with substrate on one side of the acyl chain and one side of the alcohol part. In this region it is preferred to substitute an amino acid of the parent lipase with a more positive amino acid or with a less hydrophobic amino acid. Amino acid residues corresponding to the following positions are comprised by Region II: 202 to 211 and 249 to 269. The following positions are of particular interest: 202, 210, 211, 253, 254, 255, 256, 259. In particular the following substitutions have been identified: X202G, X210K/W/A, X255Y/V/A, X256K/R and X259G/M/Q/V.

In a preferred embodiment the parent lipase has at least 80%, such as 85% or 90%, such as at least 95% or 96% or 97% or 98% or 99%, identity to SEQ ID NO:2. In a most preferred embodiment the parent lipase is identical to SEQ ID NO: 2.

### Substitutions in Region III

Region III consists of amino acid residues that form a flexible structure and thus allowing the substrate to get into the active site. In this region it is preferred to substitute an amino acid of the parent lipase with a more positive amino acid or a less hydrophobic amino acid. Amino acid residues corresponding to the following positions are comprised by Region III: 82 to 102. The following positions are of particular interest: 83, 86, 87, 90, 91, 95, 96, 99. In particular the following substitutions have been identified: X83T, X86V and X90A/R.

In a preferred embodiment the parent lipase has at least 80%, such as 85% or 90%, such as at least 95% or 96% or 97% or 98% or 99%, identity to SEQ ID NO:2. In a most preferred embodiment the parent lipase is identical to SEQ ID NO: 2.

### Substitutions in Region IV

Region IV consists of amino acid residues that bind electrostatically to a surface. In this region it is preferred to substitute an amino acid of the parent lipase with a more positive amino acid. Amino acid residues corresponding to the following positions are comprised by Region IV: 27 and 54 to 62. The following positions are of particular interest: 27, 56, 57, 58, 60. In particular the following substitutions have been identified: X27R, X58N/AG/T/P and X60V/S/G/N/R/K/A/L.

In a preferred embodiment the parent lipase has at least 80%, such as 85% or 90%, such as at least 95% or 96% or 97% or 98% or 99%, identity to SEQ ID NO:2. In a most preferred embodiment the parent lipase is identical to SEQ ID NO: 2.

### Amino Acids at Other Positions

The parent lipase may optionally comprise substitutions of other amino acids, particularly less than 10 or less than 5 such substitutions. Examples are substitutions corresponding to one or more of the positions 24, 37, 38, 46, 74, 81, 83, 115, 127, 131, 137, 143, 147, 150, 199, 200, 203, 206, 211, 263, 264, 265, 267 and 269 of the parent lipase. In a particular embodiment there is a substitution in at least one of the positions corresponding to position 81, 143, 147, 150 and 249. In a preferred embodiment the at least one substitution is selected from the group consisting of X81Q/E, X143S/C/N/D/A, X147M/Y, X150G/K and X249R/I/L.

The variant may comprise substitutions outside the defined Regions I to IV, the number of substitutions outside of the defined Regions I to IV is preferably less than six, or less than five, or less than four, or less than three, or less than two, such as five, or four, or three, or two or one. Alternatively, the variant does not comprise any substitution outside of the defined Regions I to IV.

Further substitutions may, e.g., be made according to principles known in the art, e.g. substitutions described in WO 92/05249, WO 94/25577, WO 95/22615, WO 97/04079 and WO 97/07202.

### Parent Lipase Variants

In one aspect, said variant, when compared to said parent, comprising a total of at least three substitutions, said substitutions being selected from one or more of the following groups of substitutions:
a) at least two, or at least three, or at least four, or at least five, or at least six, such as two, three, four, five or six, substitutions in Region I,
b) at least one, at least two, or at least three, or at least four, or at least five, or at least six, such as one, two, three, four, five or six, substitution in Region II,
c) at least one, at least two, or at least three, or at least four, or at least five, or at least six, such as one, two, three, four, five or six, substitution in Region III,
d) and/or at least one, at least two, or at least three, or at least four, or at least five, or at least six, such as one, two, three, four, five or six, substitution in Region IV.

The variant may comprise substitutions, compared to the variant's parent, corresponding to those substitutions listed below in Table 1.

TABLE 1
Some particular variants.

| Region I | Region II | Region III | Region IV | Outside regions |
|---|---|---|---|---|
| X4V + X227G + X231R + X233R | X210K + X256K | X83T + X86V | X58A + X60S | X150G |
| X227G + X231R + X233R | X256K | X86V | X58N + X60S | X150G |
| X231R + X233R | X255Y | | | |
| X231R + X233R | X202G | | | |
| X227G + X231R + X233R | X256K | X86V | | |
| X4V + X231R + X233R | | | X58N + X60S | |
| X231R + X233R | | X90R | X58N + X60S | |
| X231R + X233R | X255V | X90A | | |
| X227G + X231R + X233R | X256K | X86V | X58N + X60S | X150G |
| X231R + X233R | X211L | | X58N + X60S | X147M |
| X231R + X233R | | | | X150K |

In a further particular embodiment the parent lipase is identical to SEQ ID NO:2, and the variants of Table 1 will thus be:

TABLE 2
Some particular variants of SEQ ID NO: 2

| Region I | Region II | Region III | Region IV | Outside regions |
|---|---|---|---|---|
| Q4V + L227G + T231R + N233R | E210K + P256K | S83T + I86V | S58A + V60S | A150G |
| L227G + T231R + N233R | P256K | I86V | S58N + V60S | A150G |
| T231R + N233R | I255Y | | | |
| T231R + N233R | I202G | | | |
| L227G + T231R + N233R | P256K | I86V | | |
| Q4V + T231R + N233R | | | S58N + V60S | |
| T231R + N233R | | I90R | S58N + V60S | |
| T231R + N233R | I255V | I90A | | |
| L227G + T231R + N233R | P256K | I86V | S58N + V60S | A150G |
| T231R + N233R | F211L | | S58N + V60S | L147M |
| T231R + N233R | | | | A150K |

### Nomenclature for Amino Acid Modifications

In describing lipase variants according to the invention, the following nomenclature is used for ease of reference: Original amino acid(s):position(s):substituted amino acid(s)

According to this nomenclature, for instance the substitution of glutamic acid for glycine in position 195 is shown as G195E. A deletion of glycine in the same position is shown as G195*, and insertion of an additional amino acid residue such as lysine is shown as G195GK. Where a specific lipase contains a “deletion” in comparison with other lipases and an insertion is made in such a position this is indicated as *36D for insertion of an aspartic acid in position 36. Multiple mutations are separated by pluses, i.e.: R170Y+G195E, representing mutations in positions 170 and 195 substituting tyrosine and glutamic acid for arginine and glycine, respectively.

X231 indicates the amino acid in a parent polypeptide corresponding to position 231, when applying the described alignment procedure. X231R indicates that the amino acid is replaced with R. For SEQ ID NO:2 X is T, and X231R thus indicates a substitution of T in position 231 with R. Where the amino acid in a position (e.g. 231) may be substituted by another amino acid selected from a group of amino acids, e.g. the group consisting of R and P and Y, this will be indicated by X231R/P/Y.

In all cases, the accepted IUPAC single letter or triple letter amino acid abbreviation is employed.

### Amino Acid Grouping

In this specification, amino acids are classified as negatively charged, positively charged or electrically neutral according to their electric charge at pH 10. Thus, negative amino acids are E, D, C (cysteine) and Y, particularly E and D. Positive amino acids are R, K and H, particularly R and K. Neutral amino acids are G, A, V, L, I, P, F, W, S, T, M, N, Q and C when forming part of a disulfide bridge. A substitution with another amino acid in the same group (negative, positive or neutral) is termed a conservative substitution.

The neutral amino acids may be divided into hydrophobic or non-polar (G. A, V, L, I, P, F, W and C as part of a disulfide bridge) and hydrophilic or polar (S, T, M, N, Q).

### Amino Acid Identity

The relatedness between two amino acid sequences or between two nucleotide sequences is described by the parameter “identity”.

For purposes of the present invention, the alignment of two amino acid sequences is determined by using the Needle program from the EMBOSS package (http://emboss.org) version 2.8.0. The Needle program implements the global alignment algorithm described in Needleman, S. B. and Wunsch, C. D. (1970) J. Mol. Biol. 48, 443-453. The substitution matrix used is BLOSUM62, gap opening penalty is 10, and gap extension penalty is 0.5.

The degree of identity between an amino acid sequence of the present invention (“invention sequence”; e.g. amino acids 1 to 269 of SEQ ID NO:2) and a different amino acid sequence (“foreign sequence”) is calculated as the number of exact matches in an alignment of the two sequences, divided by the length of the “invention sequence” or the length of the “foreign sequence”, whichever is the shortest. The result is expressed in percent identity.

An exact match occurs when the “invention sequence” and the “foreign sequence” have identical amino acid residues in the same positions of the overlap. The length of a sequence is the number of amino acid residues in the sequence (e.g. the length of SEQ ID NO:2 is 269).

The parent lipase has an amino acid identity of at least 50% with the T lanuginosus lipase (SEQ ID NO: 2), particularly at least 55%, at least 60%, at least 75%, at least 85%, at least 90%, more than 95% or more than 98%. In a particular embodiment the parent lipase is identical to the T lanuginosus lipase (SEQ ID NO:2).

The above procedure may be used for calculation of identity as well as homology and for alignment. In the context of the present invention homology and alignment has been calculated as described below.

### Homology and Alignment

For purposes of the present invention, the degree of homology may be suitably determined by means of computer programs known in the art, such as GAP provided in the GCG program package (Program Manual for the Wisconsin Package, Version 8, August 1994, Genetics Computer Group, 575 Science Drive, Madison, Wis., USA 53711) (Needleman, S. B. and Wunsch, C. D., (1970), Journal of Molecular Biology, 48, 443-45), using GAP with the following settings for polypeptide sequence comparison: GAP creation penalty of 3.0 and GAP extension penalty of 0.1.

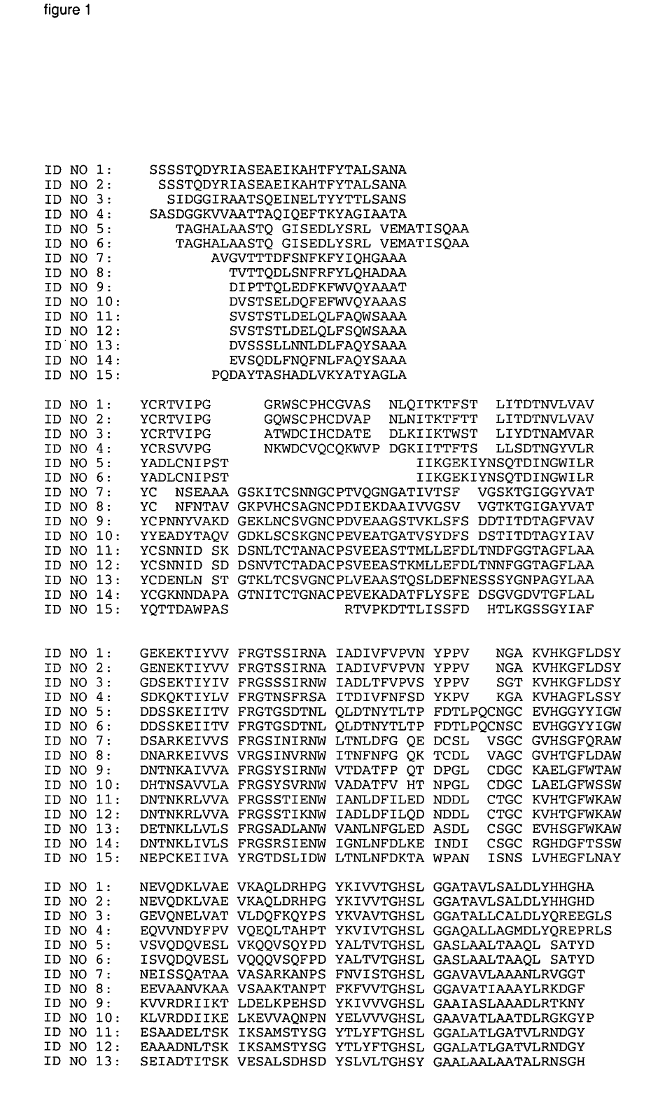

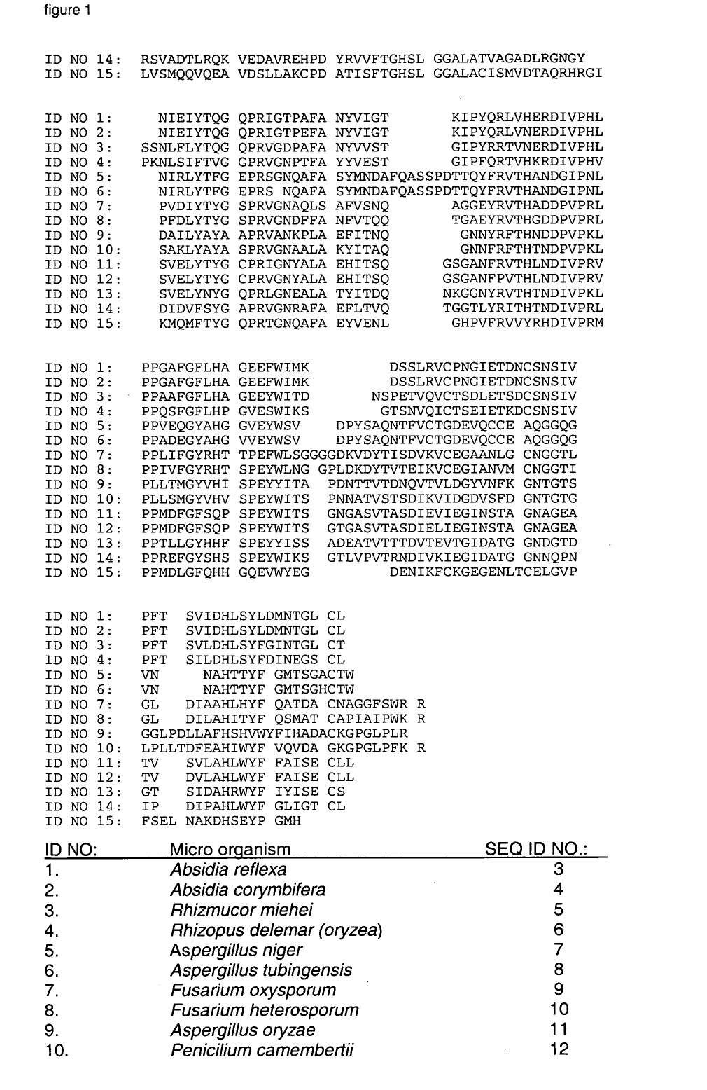

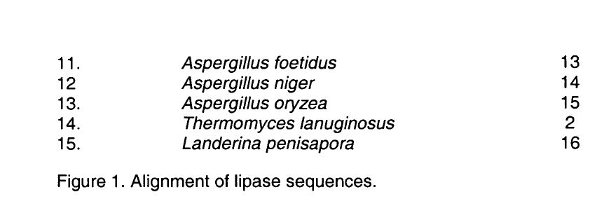

In the present invention, corresponding (or homologous) positions in the lipase sequences of Absidia reflexa, Absidia corymbefera, Rhizmucor miehei, Rhizopus delemar, Aspergillus niger, Aspergillus tubigensis, Fusarium oxysporum, Fusarium heterosporum, Aspergillus oryzea, Penicilium camembertii, Aspergillus foetidus, Aspergillus niger, Thermomyces lanoginosus (synonym: Humicola lanuginose) and Landerina penisapora are defined by the alignment shown in FIG. 1.

To find the homologous positions in lipase sequences not shown in the alignment, the sequence of interest is aligned to the sequences shown in FIG. 1. The new sequence is aligned to the present alignment in FIG. 1 by using the GAP alignment to the most homologous sequence found by the GAP program. GAP is provided in the GCG program package (Program Manual for the Wisconsin Package, Version 8, August 1994, Genetics Computer Group, 575 Science Drive, Madison, Wis., USA 53711) (Needleman, S. B. and Wunsch, C. D., (1970), Journal of Molecular Biology, 48, 443-45). The following settings are used for polypeptide sequence comparison: GAP creation penalty of 3.0 and GAP extension penalty of 0.1.

The parent lipase has a homology of at least 50% with the T lanuginosus lipase (SEQ ID NO: 2), particularly at least 55%, at least 60%, at least 75%, at least 85%, at least 90%, more than 95% or more than 98%. In a particular embodiment the parent lipase is identical to the T lanuginosus lipase (SEQ ID NO:2).

### Hybridization

The present invention also relates to isolated polypeptides having lipase activity which are encoded by polynucleotides which hybridize under very low stringency conditions, preferably low stringency conditions, more preferably medium stringency conditions, more preferably medium-high stringency conditions, even more preferably high stringency conditions, and most preferably very high stringency conditions with (i) nucleotides 178 to 660 of SEQ ID NO: 1, (ii) the cDNA sequence contained in nucleotides 178 to 660 of SEQ ID NO: 1, (iii) a subsequence of (i) or (ii), or (iv) a complementary strand of (i), (ii), or (iii) (J. Sambrook, E. F. Fritsch, and T. Maniatus, 1989, Molecular Cloning, A Laboratory Manual, 2d edition, Cold Spring Harbor, N.Y.). A subsequence of SEQ ID NO: 1 contains at least 100 contiguous nucleotides or preferably at least 200 contiguous nucleotides. Moreover, the subsequence may encode a polypeptide fragment which has lipase activity.

For long probes of at least 100 nucleotides in length, very low to very high stringency conditions are defined as prehybridization and hybridization at 42° C. in 5×SSPE, 0.3% SDS, 200 ug/ml sheared and denatured salmon sperm DNA, and either 25% formamide for very low and low stringencies, 35% formamide for medium and medium-high stringencies, or 50% formamide for high and very high stringencies, following standard Southern blotting procedures for 12 to 24 hours optimally.

For long probes of at least 100 nucleotides in length, the carrier material is finally washed three times each for 15 minutes using 2×SSC, 0.2% SDS preferably at least at 45° C. (very low stringency), more preferably at least at 50° C. (low stringency), more preferably at least at 55° C. (medium stringency), more preferably at least at 60° C. (medium-high stringency), even more preferably at least at 65° C. (high stringency), and most preferably at least at 70° C. (very high stringency).

### DNA Sequence, Expression Vector, Host Cell, Production of Lipase

The invention provides a DNA sequence encoding the lipase of the invention, an expression vector harboring the DNA sequence, and a transformed host cell containing the DNA sequence or the expression vector. These may be obtained by methods known in the art.

The invention also provides a method of producing the lipase by culturing the transformed host cell under conditions conducive for the production of the lipase and recovering the lipase from the resulting broth. The method may be practiced according to principles known in the art.

### Lipase Activity

Lipase Activity on Tributyrin at Neutral pH (LU)

A substrate for lipase is prepared by emulsifying tributyrin (glycerin tributyrate) using gum Arabic as emulsifier. The hydrolysis of tributyrin at 30° C. at pH 7 or 9 is followed in a pH-stat titration experiment. One unit of lipase activity (1 LU) equals the amount of enzyme capable of releasing 1 micro mol butyric acid/min at pH 7.

Benefit Risk

The Benefit Risk factor describing the performance compared to the reduced risk for odour smell is defined as: BR=RP_{avg}/R. Lipase variants described herein may have BRs greater than 1, greater than 1.1, or even greater than 1 to about 1000.

Average Relative Performance

The procedure for calculating average relative performance (RPavg) is found in Example 5 of the present specification. Lipase variants described herein may have (RPavg) of at least 0.8, at least 1.1, at least 1.5, or even at least 2 to about 1000.

### Suitable Photobleaches

Suitable photobleaches include catalytic photobleaches and photo-initiators. Suitable catalytic photobleaches include catalytic photobleaches selected from the group consisting of water soluble phthalocyanines of the formula:

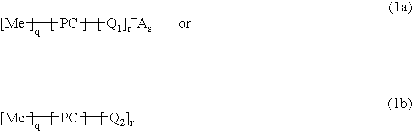

in which:
PC is the phthalocyanine ring system;
Me is Zn; Fe(II); Ca; Mg; Na; K; Al-Z₁; Si(IV); P(V); Ti(IV); Ge(IV); Cr(VI); Ga(III); Zr(IV); In(III); Sn(IV) or Hf(VI);
Z₁ is a halide; sulfate; nitrate; carboxylate; alkanolate; or hydroxyl ion;
q is 0; 1 or 2;
r is 1 to 4;
Q₁, is a sulfo or carboxyl group; or a radical of the formula

—SO₂X₂—R₁—X₃⁺; —O—R₁—X₃⁺; or —(CH₂),—Y₁⁺;
in which
   R₁ is a branched or unbranched C₁-C₈ alkylene; or 1,3- or 1,4-phenylene;
   X₂ is —NH—; or —N—C₁-C₅ alkyl;
   X₃⁺ is a group of the formula

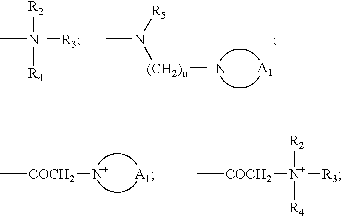

or, in the case where R₁=C₁-C₈alkylene, also a group of the formula

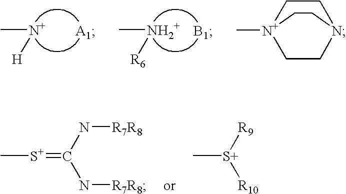

Y₁⁺ is a group of the formula

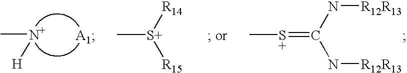

t is 0 or 1

where in the above formulae
R₂ and R₃ independently of one another are C₁-C₆ alkyl
R₄ is C₁-C₅ alkyl; C₅-C₇ cycloalkyl or NR₇R₈;
R₅ and R₆ independently of one another are C₁-C₅ alkyl;
R₇ and R₈ independently of one another are hydrogen or C₁-C₅ alkyl;
R₉ and R₁₀ independently of one another are unsubstituted C₁-C₆ alkyl or C₁-C₆ alkyl substituted by hydroxyl, cyano, carboxyl, carb-C₁-C₆ alkoxy, C₁-C₆ alkoxy, phenyl, naphthyl or pyridyl;
u is from 1 to 6;
A₁ is a unit which completes an aromatic 5- to 7-membered nitrogen heterocycle, which may where appropriate also contain one or two further nitrogen atoms as ring members, and
B₁ is a unit which completes a saturated 5- to 7-membered nitrogen heterocycle, which may where appropriate also contain 1 to 2 nitrogen, oxygen and/or sulfur atoms as ring members;
Q₂ is hydroxyl; C₁-C₂₂ alkyl; branched C₃-C₂₂ alkyl; C₂-C₂₂ alkenyl; branched C₃-C₂₂ alkenyl and mixtures thereof; C₁-C₂₂ alkoxy; a sulfo or carboxyl radical; a radical of the formula

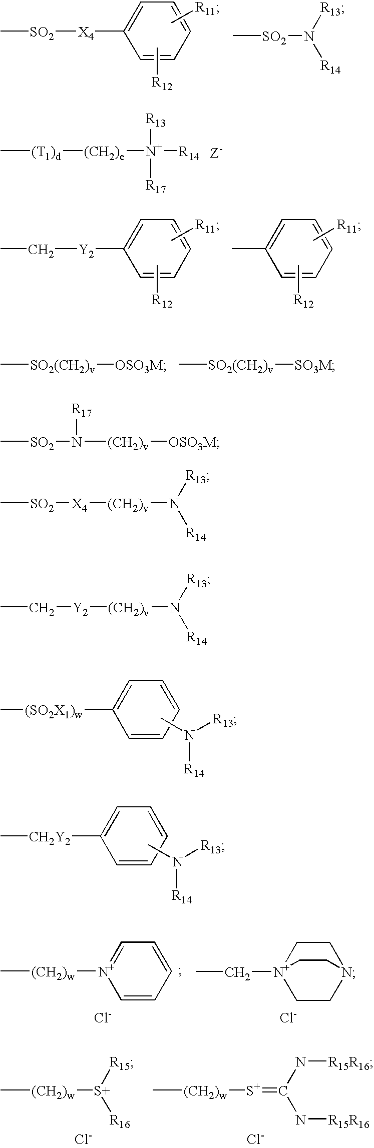

a branched alkoxy radical of the formula

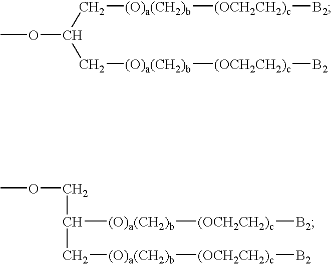

an alkylethyleneoxy unit of the formula

-(T₁)_{d}-(CH₂)_{b}(OCH₂CH₂)ₐ—B₃

or an ester of the formula

COOR₁₈

in which
B₂ is hydrogen; hydroxyl; C₁-C₃₀ alkyl; C₁-C₃₀ alkoxy; —CO₂H; —CH₂COOH; —SO₃-M₁; —OSO₃-M₁; —PO₃²⁻M₁; —OPO₃²⁻M₁; and mixtures thereof;
B₃ is hydrogen; hydroxyl; —COOH; —SO₃-M₁; —OSO₃M₁ or C₁-C₆ alkoxy;
M₁ is a water-soluble cation;
T₁ is —O—; or —NH—;
X₁ and X₄ independently of one another are —O—; —NH— or —N—C₁-C₅alkyl;
R₁₁ and R₁₂ independently of one another are hydrogen; a sulfo group and salts thereof; a carboxyl group and salts thereof or a hydroxyl group; at least one of the radicals R₁₁ and R₁₂ being a sulfo or carboxyl group or salts thereof,
Y₂ is —O—; —S—; —NH— or —N—C₁-C₅alkyl;
R₁₃ and R₁₄ independently of one another are hydrogen; C₁-C₆ alkyl; hydroxy-C₁-C₆ alkyl; cyano-C₁-C₆ alkyl; sulfo-C₁-C₆ alkyl; carboxy or halogen-C₁-C₆ alkyl; unsubstituted phenyl or phenyl substituted by halogen, C₁-C₄ alkyl or C₁-C₄ alkoxy; sulfo or carboxyl or R₁₃ and R₁₄ together with the nitrogen atom to which they are bonded form a saturated 5- or 6-membered heterocyclic ring which may additionally also contain a nitrogen or oxygen atom as a ring member;
R₁₅ and R₁₆ independently of one another are C₁-C₆ alkyl or aryl-C₁-C₆ alkyl radicals;
R₁₇ is hydrogen; an unsubstituted C₁-C₆ alkyl or C₁-C₆ alkyl substituted by halogen, hydroxyl, cyano, phenyl, carboxyl, carb-C₁-C₆ alkoxy or C₁-C₆ alkoxy;
R₁₈ is C₁-C₂₂ alkyl; branched C₃-C₂₂ alkyl; C₁-C₂₂ alkenyl or branched C₃-C₂₂ alkenyl; C₃-C₂₂ glycol; C₁-C₂₂ alkoxy; branched C₃-C₂₂ alkoxy; and mixtures thereof;
M is hydrogen; or an alkali metal ion or ammonium ion,
Z₂⁻ is a chlorine; bromine; alkylsulfate or arylsulfate ion;
a is 0 or 1;
b is from 0 to 6;
c is from 0 to 100;
d is 0; or 1;
e is from 0 to 22;
v is an integer from 2 to 12;
w is 0 or 1; and
A⁻ is an organic or inorganic anion, and
s is equal to r in cases of monovalent anions A⁻ and less than or equal to r in cases of polyvalent anions, it being necessary for Aₛ⁻ to compensate the positive charge; where, when r is not equal to 1, the radicals Q₁ can be identical or different,
   and where the phthalocyanine ring system may also comprise further solubilising groups;

Other suitable catalytic photobleaches include xanthene dyes and mixtures thereof. In another aspect, suitable catalytic photobleaches include catalytic photobleaches selected from the group consisting of sulfonated zinc phthalocyanine, sulfonated aluminium phthalocyanine, Eosin Y, Phoxine B, Rose Bengal, C.I. Food Red 14 and mixtures thereof. In another aspect a suitable photobleach may be a mixture of sulfonated zinc phthalocyanine and sulfonated aluminium phthalocyanine, said mixture having a weight ratio of sulfonated zinc phthalocyanine to sulfonated aluminium phthalocyanine greater than 1, greater than 1 but less than about 100, or even from about 1 to about 4.

Suitable photo-initiators include photo-initiators selected from the group consisting of Aromatic 1,4-quinones such as anthraquinones and naphthaquinones; Alpha amino ketones, particularly those containing a benzoyl moiety, otherwise called alpha-amino acetophenones; Alphahydroxy ketones, particularly alpha-hydroxy acetophenones; Phosphorus-containing photoinitiators, including monoacyl, bisacyl and trisacyl phosphine oxide and sulphides; Dialkoxy acetophenones; Alpha-haloacetophenones; Trisacyl phosphine oxides; Benzoin and benzoin based photoinitiators, and mixtures thereof. In another aspect, suitable photo-initiators include photo-initiators selected from the group consisting of 2-ethyl anthraquinone; Vitamin K3; 2-sulphate-anthraquinone; 2-methyl 1-[4-phenyl]-2-morpholinopropan-1-one (Irgacure® 907); (2-benzyl-2-dimethyl amino-1-(4-morpholinophenyl)-butan-1-one (Irgacure® 369); (1-[4-(2-hydroxyethoxy)-phenyl]-2 hydroxy-2-methyl-1-propan-1-one) (Irgacure(® 2959); 1-hydroxy-cyclohexyl-phenyl-ketone (Irgacure® 184); oligo[2-hydroxy 2-methyl-1-[4(1-methyl)-phenyl]propanone (Esacure® KIP 150); 2-4-6-(trimethylbenzoyl)diphenyl-phosphine oxide, bis(2,4,6-trimethylbenzoyl)-phenyl-phosphine oxide (Irgacure® 819); (2,4,6 trimethylbenzoyl)phenyl phosphinic acid ethyl ester (Lucirin® TPO-L); and mixtures thereof.

The aforementioned photobleaches can be used in combination (any mixture of photobleaches can be used). Suitable photobleaches can be purchased from Aldrich, Milwaukee, Wis., USA; Frontier Scientific, Logan, Utah, USA; Ciba Specialty Chemicals, Basel, Switzerland; BASF, Ludwigshafen, Germany; Lamberti S.p.A, Gallarate, Italy; Dayglo Color Corporation, Mumbai, India; Organic Dyestuffs Corp., East Providence, R.I., USA; and/or made in accordance with the examples contained herein.

### Adjunct Materials

While not essential for the purposes of the present invention, the non-limiting list of adjuncts illustrated hereinafter are suitable for use in the instant compositions and may be desirably incorporated in certain embodiments of the invention, for example to assist or enhance cleaning performance, for treatment of the substrate to be cleaned, or to modify the aesthetics of the cleaning composition as is the case with perfumes, colorants, dyes or the like. The precise nature of these additional components, and levels of incorporation thereof, will depend on the physical form of the composition and the nature of the cleaning operation for which it is to be used. Suitable adjunct materials include, but are not limited to, surfactants, builders, chelating agents, dye transfer inhibiting agents, dispersants, additional enzymes, and enzyme stabilizers, catalytic materials, bleach activators, hydrogen peroxide, sources of hydrogen peroxide, preformed peracids, polymeric dispersing agents, clay soil removal/anti-redeposition agents, brighteners, suds suppressors, dyes, fabric hueing agents, perfumes, structure elasticizing agents, fabric softeners, carriers, hydrotropes, processing aids, solvents and/or pigments. In addition to the disclosure below, suitable examples of such other adjuncts and levels of use are found in U.S. Pat. Nos. 5,576,282, 6,306,812 B1 and 6,326,348 B1 that are incorporated by reference.

As stated, the adjunct ingredients are not essential to Applicants' compositions. Thus, certain embodiments of Applicants' compositions do not contain one or more of the following adjuncts materials: surfactants, builders, chelating agents, dye transfer inhibiting agents, dispersants, additional enzymes, and enzyme stabilizers, catalytic materials, bleach activators, hydrogen peroxide, sources of hydrogen peroxide, preformed peracids, polymeric dispersing agents, clay soil removal/anti-redeposition agents, brighteners, suds suppressors, dyes, perfumes, structure elasticizing agents, fabric softeners, carriers, hydrotropes, processing aids, solvents and/or pigments. However, when one or more adjuncts are present, such one or more adjuncts may be present as detailed below:

Bleaching Agents—The cleaning compositions of the present invention may comprise one or more bleaching agents. Suitable bleaching agents other than bleaching catalysts include photobleaches, bleach activators, hydrogen peroxide, sources of hydrogen peroxide, pre-formed peracids and mixtures thereof. In general, when a bleaching agent is used, the compositions of the present invention may comprise from about 0.1% to about 50% or even from about 0.1% to about 25% bleaching agent by weight of the subject cleaning composition. Examples of suitable bleaching agents include:
(1) preformed peracids: Suitable preformed peracids include, but are not limited to, compounds selected from the group consisting of percarboxylic acids and salts, percarbonic acids and salts, perimidic acids and salts, peroxymonosulfuric acids and salts, for example, Oxzone®, and mixtures thereof. Suitable percarboxylic acids include hydrophobic and hydrophilic peracids having the formula R—(C═O)O—O-M wherein R is an alkyl group, optionally branched, having, when the peracid is hydrophobic, from 6 to 14 carbon atoms, or from 8 to 12 carbon atoms and, when the peracid is hydrophilic, less than 6 carbon atoms or even less than 4 carbon atoms; and M is a counterion, for example, sodium, potassium or hydrogen;
(2) sources of hydrogen peroxide, for example, inorganic perhydrate salts, including alkali metal salts such as sodium salts of perborate (usually mono- or tetra-hydrate), percarbonate, persulphate, perphosphate, persilicate salts and mixtures thereof. In one aspect of the invention the inorganic perhydrate salts are selected from the group consisting of sodium salts of perborate, percarbonate and mixtures thereof. When employed, inorganic perhydrate salts are typically present in amounts of from 0.05 to 40 wt %, or 1 to 30 wt % of the overall composition and are typically incorporated into such compositions as a crystalline solid that may be coated. Suitable coatings include, inorganic salts such as alkali metal silicate, carbonate or borate salts or mixtures thereof, or organic materials such as water-soluble or dispersible polymers, waxes, oils or fatty soaps; and
(3) bleach activators having R—(C═O)-L wherein R is an alkyl group, optionally branched, having, when the bleach activator is hydrophobic, from 6 to 14 carbon atoms, or from 8 to 12 carbon atoms and, when the bleach activator is hydrophilic, less than 6 carbon atoms or even less than 4 carbon atoms; and L is leaving group. Examples of suitable leaving groups are benzoic acid and derivatives thereof—especially benzene sulphonate. Suitable bleach activators include dodecanoyl oxybenzene sulphonate, decanoyl oxybenzene sulphonate, decanoyl oxybenzoic acid or salts thereof, 3,5,5-trimethyl hexanoyloxybenzene sulphonate, tetraacetyl ethylene diamine (TAED) and nonanoyloxybenzene sulphonate (NOBS). Suitable bleach activators are also disclosed in WO 98/17767. While any suitable bleach activator may be employed, in one aspect of the invention the subject cleaning composition may comprise NOBS, TAED or mixtures thereof.

When present, the peracid and/or bleach activator is generally present in the composition in an amount of from about 0.1 to about 60 wt %, from about 0.5 to about 40 wt % or even from about 0.6 to about 10 wt % based on the composition. One or more hydrophobic peracids or precursors thereof may be used in combination with one or more hydrophilic peracid or precursor thereof.

The amounts of hydrogen peroxide source and peracid or bleach activator may be selected such that the molar ratio of available oxygen (from the peroxide source) to peracid is from 1:1 to 35:1, or even 2:1 to 10:1.

Surfactants—The cleaning compositions according to the present invention may comprise a surfactant or surfactant system wherein the surfactant can be selected from nonionic surfactants, anionic surfactants, cationic surfactants, ampholytic surfactants, zwitterionic surfactants, semi-polar nonionic surfactants and mixtures thereof. When present, surfactant is typically present at a level of from about 0.1% to about 60%, from about 1% to about 50% or even from about 5% to about 40% by weight of the subject composition.

Builders—The cleaning compositions of the present invention may comprise one or more detergent builders or builder systems. When a builder is used, the subject composition will typically comprise at least about 1%, from about 5% to about 60% or even from about 10% to about 40% builder by weight of the subject composition. Builders include, but are not limited to, the alkali metal, ammonium and alkanolammonium salts of polyphosphates, alkali metal silicates, alkaline earth and alkali metal carbonates, aluminosilicate builders and polycarboxylate compounds, ether hydroxypolycarboxylates, copolymers of maleic anhydride with ethylene or vinyl methyl ether, 1,3,5-trihydroxy benzene-2,4,6-trisulphonic acid, and carboxymethyloxysuccinic acid, the various alkali metal, ammonium and substituted ammonium salts of polyacetic acids such as ethylenediamine tetraacetic acid and nitrilotriacetic acid, as well as polycarboxylates such as mellitic acid, succinic acid, citric acid, oxydisuccinic acid, polymaleic acid, benzene 1,3,5-tricarboxylic acid, carboxymethyloxysuccinic acid, and soluble salts thereof.

Chelating Agents—The cleaning compositions herein may contain a chelating agent. Suitable chelating agents include copper, iron and/or manganese chelating agents and mixtures thereof. When a chelating agent is used, the subject composition may comprise from about 0.005% to about 15% or even from about 3.0% to about 10% chelating agent by weight of the subject composition.

Dye Transfer Inhibiting Agents—The cleaning compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

Brighteners—The cleaning compositions of the present invention can also contain additional components that may tint articles being cleaned, such as fluorescent brighteners. Suitable fluorescent brightener levels include lower levels of from about 0.01, from about 0.05, from about 0.1 or even from about 0.2 wt % to upper levels of 0.5 or even 0.75 wt %.

Dispersants—The compositions of the present invention can also contain dispersants. Suitable water-soluble organic materials include the homo- or co-polymeric acids or their salts, in which the polycarboxylic acid comprises at least two carboxyl radicals separated from each other by not more than two carbon atoms.

Additional Enzymes—The cleaning compositions can comprise one or more enzymes which provide cleaning performance and/or fabric care benefits. Examples of suitable enzymes include, but are not limited to, hemicellulases, peroxidases, proteases, cellulases, xylanases, lipases, phospholipases, esterases, cutinases, pectinases, mannanases, pectate lyases, keratinases, reductases, oxidases, phenoloxidases, lipoxygenases, ligninases, pullulanases, tannases, pentosanases, malanases, β-glucanases, arabinosidases, hyaluronidase, chondroitinase, laccase, and amylases, or mixtures thereof. A typical combination is an enzyme cocktail that may comprise, for example, a protease and lipase in conjunction with amylase. When present in a cleaning composition, the aforementioned additional enzymes may be present at levels from about 0.00001% to about 2%, from about 0.0001% to about 1% or even from about 0.001% to about 0.5% enzyme protein by weight of the composition.

Enzyme Stabilizers—Enzymes for use in detergents can be stabilized by various techniques. The enzymes employed herein can be stabilized by the presence of water-soluble sources of calcium and/or magnesium ions in the finished compositions that provide such ions to the enzymes. In case of aqueous compositions comprising protease, a reversible protease inhibitor, such as a boron compound, can be added to further improve stability.

Catalytic Metal Complexes—Applicants' cleaning compositions may include catalytic metal complexes. One type of metal-containing bleach catalyst is a catalyst system comprising a transition metal cation of defined bleach catalytic activity, such as copper, iron, titanium, ruthenium, tungsten, molybdenum, or manganese cations, an auxiliary metal cation having little or no bleach catalytic activity, such as zinc or aluminum cations, and a sequestrate having defined stability constants for the catalytic and auxiliary metal cations, particularly ethylenediaminetetraacetic acid, ethylenediaminetetra(methylenephosphonic acid) and water-soluble salts thereof. Such catalysts are disclosed in U.S. Pat. No. 4,430,243.

If desired, the compositions herein can be catalyzed by means of a manganese compound. Such compounds and levels of use are well known in the art and include, for example, the manganese-based catalysts disclosed in U.S. Pat. No. 5,576,282.

Cobalt bleach catalysts useful herein are known, and are described, for example, in U.S. Pat. No. 5,597,936; U.S. Pat. No. 5,595,967. Such cobalt catalysts are readily prepared by known procedures, such as taught for example in U.S. Pat. No. 5,597,936, and U.S. Pat. No. 5,595,967.

Compositions herein may also suitably include a transition metal complex of ligands such as bispidones (WO 05/042532 A1) and/or macropolycyclic rigid ligands—abbreviated as “MRLs”. As a practical matter, and not by way of limitation, the compositions and processes herein can be adjusted to provide on the order of at least one part per hundred million of the active MRL species in the aqueous washing medium, and will typically provide from about 0.005 ppm to about 25 ppm, from about 0.05 ppm to about 10 ppm, or even from about 0.1 ppm to about 5 ppm, of the MRL in the wash liquor.

Suitable transition-metals in the instant transition-metal bleach catalyst include, for example, manganese, iron and chromium. Suitable MRLs include 5,12-diethyl-1,5,8,12-tetraazabicyclo[6.6.2]hexadecane.

Suitable transition metal MRLs are readily prepared by known procedures, such as taught for example in WO 00/32601, and U.S. Pat. No. 6,225,464.

Solvents—Suitable solvents include water and other solvents such as lipophilic fluids. Examples of suitable lipophilic fluids include siloxanes, other silicones, hydrocarbons, glycol ethers, glycerine derivatives such as glycerine ethers, perfluorinated amines, perfluorinated and hydrofluoroether solvents, low-volatility nonfluorinated organic solvents, diol solvents, other environmentally-friendly solvents and mixtures thereof.

### Processes of Making Compositions

The compositions of the present invention can be formulated into any suitable form and prepared by any process chosen by the formulator, non-limiting examples of which are described in Applicants' examples and in U.S. Pat. No. 4,990,280; U.S. 20030087791A1; U.S. 20030087790A1; U.S. 20050003983A1; U.S. 20040048764A1; U.S. Pat. No. 4,762,636; U.S. Pat. No. 6,291,412; U.S. 20050227891A1; EP 1070115A2; U.S. Pat. No. 5,879,584; U.S. Pat. No. 5,691,297; U.S. Pat. No. 5,574,005; U.S. Pat. No. 5,569,645; U.S. Pat. No. 5,565,422; U.S. Pat. No. 5,516,448; U.S. Pat. No. 5,489,392; U.S. Pat. No. 5,486,303 all of which are incorporated herein by reference.

### Method of Use

The present invention includes a method for cleaning and /or treating a situs inter alia a surface or fabric. Such method includes the steps of contacting an embodiment of Applicants' cleaning composition, in neat form or diluted in a wash liquor, with at least a portion of a surface or fabric then optionally rinsing such surface or fabric. The surface or fabric may be subjected to a washing step prior to the aforementioned rinsing step. For purposes of the present invention, washing includes but is not limited to, scrubbing, and mechanical agitation. As will be appreciated by one skilled in the art, the cleaning compositions of the present invention are ideally suited for use in laundry applications. Accordingly, the present invention includes a method for laundering a fabric. The method comprises the steps of contacting a fabric to be laundered with a said cleaning laundry solution comprising at least one embodiment of Applicants' cleaning composition, cleaning additive or mixture thereof. The fabric may comprise most any fabric capable of being laundered in normal consumer use conditions. The solution preferably has a pH of from about 8 to about 10.5. The compositions may be employed at concentrations of from about 500 ppm to about 15,000 ppm in solution. The water temperatures typically range from about 5° C. to about 90° C. The water to fabric ratio is typically from about 1:1 to about 30:1.

### EXAMPLES

### Lipase Variants Examples

Chemicals used as buffers and substrates are commercial products of at least reagent grade.

Media and Solutions: LAS (Surfac PS™) and Zeolite A (Wessalith P™). Other ingredients used are standard laboratory reagents.

Materials: EMPA221 from EMPA St. Gallen, Lerchfeldstrasse 5, CH-9014 St. Gallen, Switzerland

### Example 1

### Production of Enzyme

A plasmid containing the gene encoding the lipase is constructed and transformed into a suitable host cell using standard methods of the art.

Fermentation is carried out as a fed-batch fermentation using a constant medium temperature of 34° C. and a start volume of 1.2 liter. The initial pH of the medium is set to 6.5. Once the pH has increased to 7.0 this value is maintained through addition of 10% H3PO4. The level of dissolved oxygen in the medium is controlled by varying the agitation rate and using a fixed aeration rate of 1.0 liter air per liter medium per minute. The feed addition rate is maintained at a constant level during the entire fed-batch phase.

The batch medium contained maltose syrup as carbon source, urea and yeast extract as nitrogen source and a mixture of trace metals and salts. The feed added continuously during the fed-batch phase contains maltose syrup as carbon source whereas yeast extract and urea is added in order to assure a sufficient supply of nitrogen.

Purification of the lipase may be done by use of standard methods known in the art, e.g. by filtering the fermentation supernatant and subsequent hydrophobic chromatography and anion exchange, e.g. as described in EP 0 851 913, Example 3.

### Example 2

### AMSA—Automated Mechanical Stress Assay—for Calculation of Relative Performance (RP)

The enzyme variants of the present application are tested using the Automatic Mechanical Stress Assay (AMSA). With the AMSA test the wash performance of a large quantity of small volume enzyme-detergent solutions can be examined. The AMSA plate has a number of slots for test solutions and a lid firmly squeezing the textile swatch to be washed against all the slot openings. During the washing time, the plate, test solutions, textile and lid are vigorously shaken to bring the test solution in contact with the textile and apply mechanical stress. For further description see WO 02/42740 especially the paragraph “Special method embodiments” at page 23-24. The containers, which contain the detergent test solution, consist of cylindrical holes (6 mm diameter, 10 mm depth) in a metal plate. The stained fabric (test material) lies on the top of the metal plate and is used as a lid and seal on the containers. Another metal plate lies on the top of the stained fabric to avoid any spillage from each container. The two metal plates together with the stained fabric are vibrated up and down at a frequency of 30 Hz with an amplitude of 2 mm.

The assay is conducted under the experimental conditions specified below:

TABLE 3

| | |
|---|---|
| Test solution | 0.5 g/l LAS |
| | 0.52 g/l Na2CO3 |
| | 1.07 g/l Zeolite A |
| | 0.52 g/l Tri sodium Citrate |
| Test solution volume | 160 micro 1 |
| pH | As is (≈9.9) |
| Wash time | 20 minutes |
| Temperature | 30° C. |
| Water hardness | 15°dH |
| | Ratio of Ca²⁺/Mg²⁺/NaHCO₃:4:1:7.5 |
| Enzyme concentration in test | 0.125, 0.25, 0.50, 1.0 mg enzyme |
| solution | protein/liter |
| Drying | Performance: After washing the |
| | textile pieces is immediately flushed |
| | in tap water and air-dried at 85 C. in 5 min |
| | Odor: After washing the textile |
| | pieces is immediately flushed in tap |
| | water and dried at room temperature |
| | (20° C.) for 2 hours |
| Test material | Cream turmeric swatch as described |
| | below (EMPA221 used as cotton |
| | textile) |

Cream-turmeric swatches are prepared by mixing 5 g of turmeric (Santa Maria, Denmark) with 100 g cream (38% fat, Arla, Denmark) at 50° C., the mixture was left at this temperature for about 20 minutes and filtered (50° C.) to remove any undissolved particles. The mixture is cooled to 20° C.) woven cotton swatches, EMPA221, are immersed in the cream-turmeric mixture and afterwards allowed to dry at room temperature over night and frozen until use. The preparation of cream-turmeric swatches is disclosed in the patent application PA 2005 00775, filed 27 May 2005.

The performance of the enzyme variant is measured as the brightness of the colour of the textile samples washed with that specific enzyme variant. Brightness can also be expressed as the intensity of the light reflected from the textile sample when ruminated with white light. When the textile is stained the intensity of the reflected light is lower, than that of a clean textile. Therefore the intensity of the reflected light can be used to measure wash performance of an enzyme variant.

Color measurements are made with a professional flatbed scanner (PFU DL2400pro), which is used to capture an image of the washed textile samples. The scans are made with a resolution of 200 dpi and with an output color depth of 24 bits. In order to get accurate results, the scanner is frequently calibrated with a Kodak reflective IT8 target.

To extract a value for the light intensity from the scanned images, a special designed software application is used (Novozymes Color Vector Analyzer). The program retrieves the 24 bit pixel values from the image and converts them into values for red, green and blue (RGB). The intensity value (Int) is calculated by adding the RGB values together as vectors and then taking the length of the resulting vector:

Int=\sqrt{r²+g²+b²}.

The wash performance (P) of the variants is calculated in accordance with the formula:

P=Int(v)−Int(r) where

### Int(v) is the light intensity value of textile surface washed with the tested enzyme and Int(r) is the light intensity value of textile surface washed without the tested enzyme.

A relative performance score is given as the result of the AMSA wash in accordance with the definition: Relative Performance scores (RP) are summing up the performances (P) of the tested enzyme variants against the reference enzyme: RP=P(test enzyme)/P(reference enzyme). RPavg indicates the average relative performance compared to the reference enzyme at all four enzyme concentrations (0.125, 0.25, 0.5, 1.0 mg ep/l)

RPavg=avg(RP(0.125), RP(0.25) RP(0.5), RP(1.0))

A variant is considered to exhibit improved wash performance, if it performs better than the reference. In the context of the present invention the reference enzyme is the lipase of SEQ ID NO:2 with the substitutions T231R+N233R.

Example 3

### GC—Gas Chromatograph—for Calculation of Risk Factor

The butyric acid release from the lipase washed swatches are measured by Solid Phase Micro Extraction Gas Chromatography (SPME-GC) using the following method. Four textile pieces (5 mm in diameter), washed in the specified solution in Table 3 containing 1 mg/l lipase, are transferred to a Gas Chromatograph (GC) vial. The samples are analysed on a Varian 3800 GC equipped with a Stabilwax—DA w/Integra-Guard column (30 m, 0.32 mm ID and 0.25 micro-m df) and a Carboxen PDMS SPME fibre (75 micro-m). Each sample was preincubated for 10 min at 40° C. followed by 20 min sampling with the SPME fibre in the head-space over the textile pieces. The sample was subsequently injected onto the column (injector temperature=250° C.). Column flow=2 ml Helium/min. Column oven temperature gradient: 0 min=40° C., 2 min=40° C., 22 min=240° C., 32 min=240° C. The butyric acid was detected by FID detection and the amount of butyric acid was calculated based on a butyric acid standard curve.

The Risk Performance Odour, R, of a lipase variant is the ratio between the amount of released butyric acid from the lipase variant washed swatch and the amount of released butyric acid from a swatch washed with the lipase of SEQ ID NO: 2 with the substitutions T231R+N233R (reference enzyme), after both values have been corrected for the amount of released butyric acid from a non-lipase washed swatch. The risk (R) of the variants is calculated in accordance with the below formula:

Odour=measured in micro g butyric acid developed at 1 mg enzyme protein/1 corrected for blank

α_{test enzyme}=Odour_{test enzyme}−Blank

α_{reference enzyme}=Odour_{reference enzyme}−Blank

R=α_{test enzyme}/α_{reference enzyme}

### A variant is considered to exhibit reduced odor compared to the reference, if the R factor is lower than 1.

### Example 4

### Activity (LU) Relative to Absorbance at 280 nm

The activity of a lipase relative to the absorbance at 280 nm is determined by the following assay LU/A280:

The activity of the lipase is determined as described above in the section Lipase activity. The absorbance of the lipase at 280 nm is measured (A280) and the ratio LU/A280 is calculated. The relative LU/A280 is calculated as the LU/A280 of the variant divided by the LU/A280 of a reference enzyme. In the context of the present invention the reference enzyme is the lipase of SEQ ID NO:2 with the substitutions T231R+N233R.

### Example 5

### BR—Benefit Risk

The Benefit Risk factor describing the performance compared to the reduced risk for odour smell is thus defined as: BR=RP_{avg}/R

### A variant is considered to exhibit improved wash performance and reduced odor, if the BR factor is higher than 1.

Applying the above methods the following results are obtained:

TABLE 4

| | | Average | | |
|---|---|---|---|---|
| | | RP | | |
| Variant | Mutations in SEQ ID NO: 2 | (RP_{avg}) | BR | LU/A280 |
| 1 | I202G + T231R + N233R | 0.84 | 1.41 | not |
| | | | | determined |
| 2 | I86V + L227G + T231R + N233R + P256K | 1.08 | 1.52 | 1700 |
| 3 | Q4V + S58N + V60S + T231R + N233R | 0.87 | 1.73 | 1950 |
| 4 | S58N + V60S + I90R + T231R + N233R | 1.06 | 1.27 | 2250 |
| 5 | I255Y + T231R + N233R | 1.19 | 1.17 | 3600 |
| 6 | I90A + T231R + N233R + I255V | 1.13 | 1.14 | 2700 |
| Reference | T231R + N233R | 1.00 | 1.00 | 3650 |
| 7 | G91A + E99K + T231R + N233R + | 0.43 | not | 850 |
| | Q249R + 270H + 271T + 272P + 273S + 274S + 275G + 276R + | | determined | |
| | 277G + 278G + 279H + 280R | | | |
| 8 | G91A + E99K + T231R, N233R + | 0.13 | not | 500 |
| | Q249R + 270H + 271T + 272P + 273S + 274S + 275G + 276R + | | determined | |
| | 277G + 278G | | | |

### The reference lipase and variants 7 and 8 in Table 4 are described in WO 2000/060063.

### Example 6

### BR—Benefit Risk

The Benefit Risk was measured for the variants listed in Table 5. The Benefit Risk factor was measured in the same way as described in Example 5 and it was found to be above 1 for all the listed variants.

TABLE 5

| Variant | Mutations in SEQ ID NO: 2 |
|---|---|
| Reference | T231R + N233R |
| 9 | L97V + T231R + N233R |
| 10 | A150G + T231R + N233R |
| 11 | I90R + T231R + N233R |
| 12 | I202V + T231R + N233R |
| 13 | L227G + T231R + N233R + P256K |
| 14 | I90A + T231R + N233R |
| 15 | T231R + N233R + I255P |
| 16 | I90V + I255V + T231R + N233R |
| 17 | F211L + L227G + T231R + N233R + I255L + P256K |
| 18 | S58N + V60S + T231R + N233R + Q249L |
| 19 | S58N + V60S + T231R + N233R + Q249I |
| 20 | A150G + L227G + T231R + N233R + P256K |
| 21 | K46L + S58N + V60S + T231R + N233R + Q249L + D254I |
| 22 | Q4L + E43T + K46I + S58N + V60S + T231R + N233R + Q249L + D254I |
| 23 | Q4L + S58N + V60S + T231R + N233R + Q249L + D254I |
| 24 | K46I + S58N + V60S + T231R + N233R + Q249L + D254L |
| 25 | K46L + S58N + V60S + K223I + T231R + N233R + D254L |
| 26 | E43T + K46I + S58N + V60S + T231R + N233R + Q249L + D254I |
| 27 | S58N + V60S + I86V + A150G + L227G + T231R + N233R + P256K |
| 28 | K24R + K46R + K74R + I86V + K98R + K127R + D137K + A150G + K223R + T231R + N233R |
| 29 | S58A + V60A + I86V + T231R + N233R |
| 30 | K24R + K46R + S58N + V60S + K74R + I86V + K98R + K127R + D137K + K223R + T231R + N233R |
| 31 | S58A + V60A + I86V + A150G + T231R + N233R |
| 32 | S58N + V60V + D62G + T231R + N233R |
| 33 | Q4V + S58N + V60S + I86V + T231R + N233R + Q249L |
| 34 | Q4V + S58N + V60S + I86V + A150G + T231R + N233R + I255V |
| 35 | Q4V + S58N + V60S + I90A + A150G + T231R + N233R + I255V |
| 36 | Y53A + S58N + V60S + T231R + N233R + P256L |
| 37 | I202L + T231R + N233R + I255A |
| 38 | S58A + V60S + I86V + A150G + L227G + T231R + N233R + P256K |
| 39 | D27R + S58N + V60S + I86V + A150G + L227G + T231R + N233R + P256K |
| 40 | V60K + I86V + A150G + L227G + T231R + N233R + P256K |
| 41 | Q4V + S58A + V60S + S83T + I86V + A150G + E210K + L227G + T231R + N233R + P256K |
| 42 | Q4V + V60K + S83T + I86V + A150G + L227G + T231R + N233R + P256K |
| 43 | D27R + V60K + I86V + A150G + L227G + T231R + N233R + P256K |
| 44 | Q4N + L6S + S58N + V60S + I86V + A150G + L227G + T231R + N233R + P256K |
| 45 | E1N + V60K + I86V + A150G + L227G + T231R + N233R + P256K |
| 46 | V60K + I86V + A150G + K223N + G225S + T231R + N233R + P256K |
| 47 | E210V + T231R + N233R + Q249R |
| 48 | S58N + V60S + E210V + T231R + N233R + Q249R |
| 49 | Q4V + V60K + I90R + T231R + N233R + I255V |
| 50 | Q4V + V60K + A150G + T231R + N233R |
| 51 | V60K + S83T + T231R + N233R |
| 52 | V60K + A150G + T231R + N233R + I255V |
| 53 | T231R + N233G + D234G |
| 54 | S58N + V60S + I86V + A150G + E210K + L227G + T231R + N233R + Q249R + P256K |
| 55 | S58N + V60S + I86V + A150G + E210K + L227G + T231R + N233R + I255A + P256K |
| 56 | S58N + V60S + I86V + A150G + G156R + E210K + L227G + T231R + N233R + I255A + P256K |
| 57 | S58T + V60K + I86V + N94K + A150G + E210V + L227G + T231R + N233R + P256K |
| 58 | S58T + V60K + I86V + D102A + A150G + L227G + T231R + N233R + P256K |
| 59 | S58T + V60K + I86V + D102A + A150G + E210V + L227G + T231R + N233R + P256K |
| 60 | S58T + V60K + S83T + I86V + N94K + A150G + E210V + L227G + T231R + N233R + P256K |
| 61 | S58A + V60S + I86V + T143S + A150G + L227G + T231R + N233R + P256K |
| 62 | G91S + D96V + D254R |
| 63 | V60L + G91M + T231W + Q249L |
| 64 | T37A + D96A + T231R + N233R + Q249G |
| 65 | E56G + E87D + T231R + N233R + D254A |
| 66 | E210K + T231R + N233R |
| 67 | D27H + E87Q + D96N + T231R + N233R + D254V |
| 68 | F181L + E210V + T231R + N233R |
| 69 | D27N + D96G + T231R + N233R |
| 70 | D96N + T231R + N233R |
| 71 | T231R + N2331 + D234G |
| 72 | S58K + V60L + E210V + Q249R |
| 73 | S58H + V60L + E210V + Q249R |
| 74 | Q4V + F55V + I86V + T231R + N233R + I255V |
| 75 | Q4V + S58T + V60K + T199L + N200A + E210K + T231R + N233R + I255A + P256K |
| 76 | Q4V + D27N + V60K + T231R + N233R |
| 77 | I90F + I202P + T231R + N233R + I255L |
| 78 | S58N + V60S + D158N + T231R + N233R |
| 79 | S58N + V60S + S115K + T231R + N233R |
| 80 | S58N + V60S + L147M + A150G + F211L + T231R + N233R |
| 81 | V60K + A150G + T231R + N233R |
| 82 | I90V + L227G + T231R + N233R + P256K |
| 83 | T231R + N233R + I255S |
| 84 | I86G + T231R + N233R |
| 85 | V60K + I202V + E210K + T231R + N233R + I255A + P256K |
| 86 | I90G + I202L + T231R + N233R + I255S |
| 87 | S58G + V60G + T231R + N233R |

### The reference lipase is described in WO 2000/060063.

### Composition Examples

Unless otherwise indicated, materials can be obtained from Aldrich, P.O. Box 2060, Milwaukee, Wis. 53201, USA.

### Examples 1-6

Granular laundry detergent compositions designed for handwashing or top-loading washing machines.

| | 1 | 2 | 3 | 4 | 5 | 6 |
|---|---|---|---|---|---|---|
| | (wt %) | (wt %) | (wt %) | (wt %) | (wt %) | (wt %) |
| Linear alkylbenzenesulfonate | 20 | 22 | 20 | 15 | 20 | 20 |
| C_{12–14} Dimethylhydroxyethyl | 0.7 | 1 | 1 | 0.6 | 0.0 | 0.7 |
| ammonium chloride | | | | | | |
| AE3S | 0.9 | 0.0 | 0.9 | 0.0 | 0.0 | 0.9 |
| AE7 | 0.0 | 0.5 | 0.0 | 1 | 3 | 1 |
| Sodium tripolyphosphate | 23 | 30 | 23 | 17 | 12 | 23 |
| Zeolite A | 0.0 | 0.0 | 0.0 | 0.0 | 10 | 0.0 |
| 1.6R Silicate (SiO₂:Na₂O | 7 | 7 | 7 | 7 | 7 | 7 |
| ratio 1.6:1) | | | | | | |
| Sodium Carbonate | 15 | 14 | 15 | 18 | 15 | 15 |
| Polyacrylate MW 4500 | 1 | 0.0 | 1 | 1 | 1.5 | 1 |
| Carboxy Methyl Cellulose | 1 | 1 | 1 | 1 | 1 | 1 |
| Savinase ® 32.89 mg/g | 0.1 | 0.07 | 0.1 | 0.1 | 0.1 | 0.1 |
| Natalase ® 8.65 mg/g | 0.1 | 0.1 | 0.1 | 0.0 | 0.1 | 0.1 |
| Lipase† 18 mg/g | 0.1 | 0.07 | 0.3 | 0.1 | 0.07 | 0.4 |
| Fluorescent Brightener 1 | 0.06 | 0.0 | 0.06 | 0.18 | 0.06 | 0.06 |
| Fluorescent Brightener 2 | 0.1 | 0.06 | 0.1 | 0.0 | 0.1 | 0.1 |
| Diethylenetriamine | 0.6 | 0.3 | 0.6 | 0.25 | 0.6 | 0.6 |
| pentacetic acid | | | | | | |
| MgSO₄ | 1 | 1 | 1 | 0.5 | 1 | 1 |
| Sodium Percarbonate | 0.0 | 5.2 | 0.1 | 0.0 | 0.0 | 0.0 |
| Sodium Perborate | 4.4 | 0.0 | 3.85 | 2.09 | 0.78 | 3.63 |
| Monohydrate | | | | | | |
| NOBS | 1.9 | 0.0 | 1.66 | — | 0.33 | 0.75 |
| TAED | 0.58 | 1.2 | 0.51 | — | 0.015 | 0.28 |
| Sulfonated zinc | 0.0030 | — | — | — | 0.0030 | — |
| phthalocyanine | | | | | | |
| Sulfonated aluminum | — | — | — | — | 0.0010 | — |
| phthalocyanine | | | | | | |
| C. I. Food Red 14 | — | 0.025 | 0.05 | — | 0.04 | 0.03 |
| 2-Ethylanthraquinone | — | — | — | 0.3 | — | — |
| Vitamin K3 | — | — | 0.25 | — | — | 0.2 |
| Sulfate/Moisture | Balance | Balance to | Balance to | Balance | Balance | Balance |
| | to 100% | 100% | 100% | to 100% | to 100% | to 100% |

### Any of the above compositions is used to launder fabrics at a concentration of 600-10,000 ppm in water, with typical median conditions of 2500 ppm, 25° C., and a 25:1 water:cloth ratio.

### Examples 7-10

Granular laundry detergent compositions designed for front-loading automatic washing machines.

| | | | | 10 |
|---|---|---|---|---|
| | 7 (wt %) | 8 (wt %) | 9 (wt %) | (wt %) |
| Linear alkylbenzenesulfonate | 8 | 7.1 | 7 | 6.5 |
| AE3S | 0 | 4.8 | 0 | 5.2 |
| Alkylsulfate | 1 | 0 | 1 | 0 |
| AE7 | 2.2 | 0 | 3.2 | 0 |
| C_{10–12} Dimethylhydroxyethylammonium | 0.75 | 0.94 | 0.98 | 0.98 |
| chloride | | | | |
| Crystalline layered silicate (δ- | 4.1 | 0 | 4.8 | 0 |
| Na₂Si₂O₅) | | | | |
| Zeolite A | 20 | 0 | 17 | 0 |
| Citric Acid | 3 | 5 | 3 | 4 |
| Sodium Carbonate | 15 | 20 | 14 | 20 |
| Silicate 2R (SiO₂:Na₂O at ratio 2:1) | 0.08 | 0 | 0.11 | 0 |
| Soil release agent | 0.75 | 0.72 | 0.71 | 0.72 |
| Acrylic Acid/Maleic Acid Copolymer | 1.1 | 3.7 | 1.0 | 3.7 |
| Carboxymethylcellulose | 0.15 | 1.4 | 0.2 | 1.4 |
| Protease (56.00 mg active/g) | 0.37 | 0.4 | 0.4 | 0.4 |
| Termamyl ® (21.55 mg active/g) | 0.3 | 0.3 | 0.3 | 0.3 |
| Lipase† (18.00 mg active/g) | 0.05 | 0.15 | 0.1 | 0.5 |
| Natalase ® (8.65 mg active/g) | 0.1 | 0.14 | 0.14 | 0.3 |
| TAED | 3.6 | 4.0 | 3.6 | 4.0 |
| Percarbonate | 13 | 13.2 | 13 | 13.2 |
| Na salt of Ethylenediamine-N,N′- | 0.2 | 0.2 | 0.2 | 0.2 |
| disuccinic acid, (S,S) isomer (EDDS) | | | | |
| Hydroxyethane di phosphonate | 0.2 | 0.2 | 0.2 | 0.2 |
| (HEDP) | | | | |
| MgSO₄ | 0.42 | 0.42 | 0.42 | 0.42 |
| Perfume | 0.5 | 0.6 | 0.5 | 0.6 |
| Suds suppressor agglomerate | 0.05 | 0.1 | 0.05 | 0.1 |
| Soap | 0.45 | 0.45 | 0.45 | 0.45 |
| Sodium sulfate | 22 | 33 | 24 | 30 |
| Sulphonated zinc phthalocyanine | 0.0007 | 0.0012 | — | — |
| C.I. Food Red 14 | — | — | 0.02 | — |
| 2-Ethylanthraquinone | — | — | — | — |
| Vitamin K3 | — | 0.07 | — | 0.1 |
| Water & Miscellaneous | Balance | Balance | Balance | Balance |
| | to 100% | to 100% | to | to |
| | | | 100% | 100% |

Any of the above compositions is used to launder fabrics at a concentration of 10,000 ppm in water, 20-90° C., and a 5:1 water:cloth ratio. The typical pH is about 10.

### Examples 11-16

### Heavy Duty Liquid Laundry Detergent Compositions

| | 11 | 12 | 13 | 14 | 15 | 16 |
|---|---|---|---|---|---|---|
| | (wt %) | (wt %) | (wt %) | (wt %) | (wt %) | (wt %) |
| AES C_{12–15} alkyl ethoxy (1.8) | 11 | 10 | 4 | 6.32 | 6.0 | 8.2 |
| sulfate | | | | | | |
| Linear alkyl benzene | 4 | 0 | 8 | 3.3 | 4.0 | 3.0 |
| sulfonate | | | | | | |
| HSAS | 0 | 5.1 | 3 | 0 | 2 | 0 |
| Sodium formate | 1.6 | 0.09 | 1.2 | 0.04 | 1.6 | 1.2 |
| Sodium hydroxide | 2.3 | 3.8 | 1.7 | 1.9 | 2.3 | 1.7 |
| Monoethanolamine | 1.4 | 1.490 | 1.0 | 0.7 | 1.35 | 1.0 |
| Diethylene glycol | 5.5 | 0.0 | 4.1 | 0.0 | 5.500 | 4.1 |
| Nonionic | 0.4 | 0.6 | 0.3 | 0.3 | 2 | 0.3 |
| Chelant | 0.15 | 0.15 | 0.11 | 0.07 | 0.15 | 0.11 |
| Citric Acid | 2.5 | 3.96 | 1.88 | 1.98 | 2.5 | 1.88 |
| C_{12–14} dimethyl | 0.3 | 0.73 | 0.23 | 0.37 | 0.3 | 0.225 |
| Amine Oxide | | | | | | |
| C_{12–18} Fatty Acid | 0.8 | 1.9 | 0.6 | 0.99 | 0.8 | 0.6 |
| Borax | 1.43 | 1.5 | 1.1 | 0.75 | 1.43 | 1.07 |
| Ethanol | 1.54 | 1.77 | 1.15 | 0.89 | 1.54 | 1.15 |
| Ethoxylated (EO₁₅) | 0.3 | 0.33 | 0.23 | 0.17 | 0.0 | 0.0 |
| tetraethylene | | | | | | |
| pentaimine¹ | | | | | | |
| Ethoxylated | 0.8 | 0.81 | 0.6 | 0.4 | 0.0 | 0.0 |
| hexamethylene | | | | | | |
| diamine² | | | | | | |
| 1,2-Propanediol | 0.0 | 6.6 | 0.0 | 3.3 | 0.0 | 0.0 |
| Protease* | 36.4 | 36.4 | 27.3 | 18.2 | 36.4 | 27.3 |
| Mannaway ®* | 1.1 | 1.1 | 0.8 | 0.6 | 1.1 | 0.8 |
| Natalase ®* | 7.3 | 7.3 | 5.5 | 3.7 | 7.3 | 5.5 |
| Lipase†* | 10 | 3.2 | 0.5 | 3.2 | 2.4 | 3.2 |
| C.I. Food Red 14 | 0.02 | — | 0.015 | — | — | 0.02 |
| Vitamin K3 | — | 0.07 | — | 0.1 | 0.04 | 0.12 |
| Water, perfume, dyes & | Balance | Balance | Balance | Balance | Balance | Balance |
| other components | | | | | | |

### Raw Materials and Notes For Composition Examples 1-16

Linear alkylbenzenesulfonate having an average aliphatic carbon chain length C₁₁-C₁₂ supplied by Stepan, Northfield, Ill., USA
C₁₂₋₁₄ Dimethylhydroxyethyl ammonium chloride, supplied by Clariant GmbH, Sulzbach, Germany
AE3S is C₁₂₋₁₅ alkyl ethoxy (3) sulfate supplied by Stepan, Northfield, Ill., USA
AE7 is C₁₂₋₁₅ alcohol ethoxylate, with an average degree of ethoxylation of 7, supplied by Huntsman, Salt Lake City, Utah, USA
Sodium tripolyphosphate is supplied by Rhodia, Paris, France
Zeolite A was supplied by Industrial Zeolite (UK) Ltd, Grays, Essex, UK
1.6R Silicate was supplied by Koma, Nestemica, Czech Republic
Sodium Carbonate was supplied by Solvay, Houston, Tex., USA
Polyacrylate MW 4500 is supplied by BASF, Ludwigshafen, Germany
Carboxy Methyl Cellulose is Finnfix® BDA supplied by CPKelco, Arnhem, Netherlands
Savinase®, Natalase®, Termamyl®, Mannaway® supplied by Novozymes, Bagsvaerd, Denmark
Lipase variant 1 to 5 described in example 5 Table 4, and combinations thereof.
Fluorescent Brightener 1 is Tinopal® AMS, Fluorescent Brightener 2 is Tinopal® CBS-X,
Sulphonated zinc phthalocyanine supplied by Ciba Specialty Chemicals, Basel, Switzerland
Diethylenetriamine pentacetic acid was supplied by Dow Chemical, Midland, Mich., USA
Sodium percarbonate supplied by Solvay, Houston, Tex., USA
Sodium perborate was supplied by Degussa, Hanau, Germany
NOBS is sodium nonanoyloxybenzenesulfonate, supplied by Eastman, Batesville, Ark., USA
TAED is tetraacetylethylenediamine, supplied under the Peractive® brand name by Clariant GmbH, Sulzbach, Germany
Soil release agent is Repel-o-tex® PF, supplied by Rhodia, Paris, France
Acrylic Acid/Maleic Acid Copolymer is molecular weight 70,000 and acrylate:maleate ratio 70:30, supplied by BASF, Ludwigshafen, Germany
Protease was FN3 supplied by Genencor International, Palo Alto, Calif., USA
Na salt of Ethylenediamine-N,N′-disuccinic acid, (S,S) isomer (EDDS) was supplied by Octel, Ellesmere Port, UK
Hydroxyethane di phosphonate (HEDP) was supplied by Dow Chemical, Midland, Mich., USA
Suds suppressor agglomerate was supplied by Dow Corning, Midland, Mich., USA
HSAS is mid-branched alkyl sulfate as disclosed in U.S. Pat. No. 6,020,303 and U.S. Pat. No. 6,060,443
C₁₂₋₁₄ dimethyl Amine Oxide was supplied by Procter & Gamble Chemicals, Cincinnati, Ohio, USA
Nonionic is preferably a C₁₂-C₁₃ ethoxylate, preferably with an average degree of ethoxylation of 9.
Protease was supplied by Genencor International, Palo Alto, Calif., USA
* Numbers quoted in mg enzyme/100 g
¹ as described in U.S. Pat. No. 4,597,898.
² available under the tradename. LUTENSIT® from BASF and such as those described in WO 01/05874
† Lipase described in the present specification.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A composition comprising a photobleach and a variant of a parent lipase, said variant, when compared to said parent, comprising a total of at least three substitutions, said substitutions being selected from one or more of the following groups of substitutions:
a.) at least two substitutions in Region I,
b) at least one substitution in Region II,
c) at least one substitution in Region III, and/or
d) at least one substitution in Region IV.

2. A detergent composition according to claim 1, wherein said substitutions in Region I comprise substitutions in the positions corresponding to the positions 231 and 233.

3. A detergent composition according to claim 2 wherein said substitutions at positions 231 and 233 are substituted with an R.

4. A detergent composition according to claim 2, wherein said variant comprises a substitution in the position corresponding to position 4 of SEQ ID NO:2.

5. A detergent composition according to claim 4, wherein said substitution in the position corresponding to position 4 of SEQ ID NO:2 is V.

6. A detergent composition according to claim 2, wherein said variant comprises a substitution in the corresponding to position 227 of SEQ ID NO:2.

7. A detergent composition according to claim 6, wherein said substitution in the position corresponding to position to position 227 of SEQ ID NO:2 is G.

8. A detergent composition according to claim 1, wherein said at least one substitution in Region II comprises a substitution selected from the group consisting of substitutions in positions corresponding to the positions 202, 211, 255 and 256.

9. A detergent composition according to claim 8, wherein said at least one substitution in Region II comprises a substitution selected from the group consisting of X202G, X211L, X255Y/V and X256K.

10. A detergent composition according to claim 1, wherein said at least one substitution in Region II comprises a substitution in the position corresponding to the position 210.

11. A detergent composition according to claim 10, wherein said substitution in the position corresponding to the position 210 comprises X210K.

12. A detergent composition according to claim 1, wherein said at least one substitution in Region III comprises a substitution selected from the group consisting of substitutions in positions corresponding to the positions 86 and 90.

13. A detergent composition according to claim 12, wherein said at least one substitution in Region III comprises a substitution selected from the group consisting of X86V and X90A/R.

14. A detergent composition according to claim 1, wherein said at least one substitution in Region III comprises a substitution in the position corresponding to the position 83.

15. A detergent composition according to claim 14, wherein said substitution in the position corresponding to the position 83 comprises X83T.

16. A detergent composition according to claim 1, wherein said at least one substitution in Region IV comprises a substitution selected from the group consisting of substitutions in positions corresponding to the positions 27, 58 and 60.

17. A detergent composition according to claim 15, wherein said at least one substitution in Region IV comprises a substitution selected from the group consisting of X27R, X58N/A/G/P/T and X60S/V/G/N/R/K/A/L.

18. A detergent composition according to claim 1, comprising at least two substitutions in Region IV corresponding to the positions 27, 58 and 60.

19. A detergent composition according to claim 1, comprising at least two substitutions in Region IV selected from the group consisting of X27R, X58N/A/G/P/T and X60S/V/G/N/R/K/A/L.

20. A detergent composition according to claim 1, wherein said variant comprises at least one substitution outside the defined Regions I to IV.

21. A detergent composition according to claim 20, wherein said at least one substitution outside the defined Regions I to IV is selected from the group consisting of substitutions in positions corresponding to position 81, 147, 150 and 249.

22. A detergent composition according to claim 20, wherein said at least one substitution outside the defined Regions I to IV is selected from the group consisting of X81Q/E, X147M/Y, X150G and X249R/I/L.

23. A detergent composition according to claim 2, wherein said parent lipase is at least 90% identical to SEQ ID NO:2.

24. A detergent composition according to claim 1 wherein the parent lipase is identical to SEQ ID NO: 2 and said variant comprises one of the following groups of substitutions:
a) T231R+N233R+I255Y
b) I202G+T231R+N233R
c) I86V+L227G+T231R+N233R+P256K
d) Q4V+S58N+V60S+T231R+N233R
e) S58N+V60S+I90R+T231R+N233R
f) I90A+T231R+N233R+I255V
g) S58N+V60S+I86V+A150G+L227G+T231R+N233R+P256K
h) S58N+V60S+L147M+F211L+T231R+N233R
i) Q4V+S58A+V60S+S83T+I86V+A150G+E210K+L227G+T231R+N233R+P256K
j) S58N+V60S+I86V+A150G+L227G+T231R+N233R+P256K.

25. A detergent composition according to claim 1 wherein the parent lipase is identical to SEQ ID NO: 2 and said variant comprises one of the following groups of substitutions:
a) Q4V+S58A+V60S+S83T+I86V+A150G+E210K+L227G+T231R+N233R+P256K
b) S58N+V60S+I86V+A150G+L227G+T231R+N233R+P256K.

26. A detergent composition according to claim 1 wherein the lipase variant is characterized in that the Benefit Risk, when measured as given in the specification, is larger than 1.

27. A detergent composition comprising a photobleach and a polypeptide having lipase activity and which further has a Average Relative Performance of at least 0.8 and a Benefit Risk of at least 1.1 at the test conditions given in the specification.

28. A composition according to claim 1 comprising 0.1 to 40% anionic surfactant.

29. A composition according to claim 28, said composition being a cleaning and/or treatment composition.

30. A composition according to claim 1, said composition comprising sulfonated zinc phthalocyanine.

31. A composition according to claim 25 comprising a mixture of sulfonated zinc phthalocyanine and sulfonated aluminium phthalocyanine, said mixture having a weight ratio of sulfonated zinc phthalocyanine to sulfonated aluminium phthalocyanine greater than 1.

32. A composition according to claim 1, said composition comprising sulfonated aluminium phthalocyanine.

33. A composition according to claim 1 wherein the photobleach comprises a xanthene dye, anthraquinone or naphthaquinone.

34. A process of cleaning and/or treating a surface or fabric comprising the steps of optionally washing and/or rinsing said surface or fabric, contacting said surface or fabric with the composition of claim 1, then optionally washing and/or rinsing said surface or fabric.

35. A composition according to claim 1, wherein said lipase variant is a variant of SEQ ID NO: 2 comprising at least one of the mutations Q4V, S58N/A/G/P/T, I90R or Q249I/L.


SEQUENCE LISTING (16 sequences)

SEQ ID NO: 1 - DNA, 807 bp, Thermomyces lanuginosus
  CDS (1)..(807)
  mat_peptide (1)..()
gaggtctcgcaggatctgtttaaccagttcaatctctttgcacagtattctgcagccgcatactgcggaaaaaacaatgatgccccagctggtacaaacattacgtgcacgggaaatgcctgccccgaggtagagaaggcggatgcaacgtttctctactcgtttgaagactctggagtgggcgatgtcaccggcttccttgctctcgacaacacgaacaaattgatcgtcctctctttccgtggctctcgttccatagagaactggatcgggaatcttaacttcgacttgaaagaaataaatgacatttgctccggctgcaggggacatgacggcttcacttcgtcctggaggtctgtagccgatacgttaaggcagaaggtggaggatgctgtgagggagcatcccgactatcgcgtggtgtttaccggacatagcttgggtggtgcattggcaactgttgccggagcagacctgcgtggaaatgggtatgatatcgacgtgttttcatatggcgccccccgagtcggaaacagggcttttgcagaattcctgaccgtacagaccggcggaacactctaccgcattacccacaccaatgatattgtccctagactcccgccgcgcgaattcggttacagccattctagcccagagtactggatcaaatctggaacccttgtccccgtcacccgaaacgatatcgtgaagatagaaggcatcgatgccaccggcggcaataaccagcctaacattccggatatccctgcgcacctatggtacttcgggttaattgggacatgtctt

SEQ ID NO: 2 - protein, 269 aa, Thermomyces lanuginosus [sequence omitted]

SEQ ID NO: 3 - protein, 265 aa, Absidia reflexa
SSSSTQDYRIASEAEIKAHTFYTALSANAYCRTVIPGGRWSCPHCGVASNLQITKTFSTLITDTNVLVAVGEKEKTIYVVFRGTSSIRNAIADIVFVPVNYPPVNGAKVHKGFLDSYNEVQDKLVAEVKAQLDRHPGYKIVVTGHSLGGATAVLSALDLYHHGHANIEIYTQGQPRIGTPAFANYVIGTKIPYQRLVHERDIVPHLPPGAFGFLHAGEEFWIMKDSSLRVCPNGIETDNCSNSIVPFTSVIDHLSYLDMNTGLCL

SEQ ID NO: 4 - protein, 264 aa, Absidia corymbifera
SSSTQDYRIASEAEIKAHTFYTALSANAYCRTVIPGGQWSCPHCDVAPNLNITKTFTTLITDTNVLVAVGENEKTIYVVFRGTSSIRNAIADIVFVPVNYPPVNGAKVHKGFLDSYNEVQDKLVAEVKAQLDRHPGYKIVVTGHSLGGATAVLSALDLYHHGHDNIEIYTQGQPRIGTPEFANYVIGTKIPYQRLVNERDIVPHLPPGAFGFLHAGEEFWIMKDSSLRVCPNGIETDNCSNSIVPFTSVIDHLSYLDMNTGLCL

SEQ ID NO: 5 - protein, 269 aa, Rhizomucor miehei
SIDGGIRAATSQEINELTYYTTLSANSYCRTVIPGATWDCIHCDATEDLKIIKTWSTLIYDTNAMVARGDSEKTIYIVFRGSSSIRNWIADLTFVPVSYPPVSGTKVHKGFLDSYGEVQNELVATVLDQFKQYPSYKVAVTGHSLGGATALLCALDLYQREEGLSSSNLFLYTQGQPRVGDPAFANYVVSTGIPYRRTVNERDIVPHLPPAAFGFLHAGEEYWITDNSPETVQVCTSDLETSDCSNSIVPFTSVLDHLSYFGINTGLCT

SEQ ID NO: 6 - protein, 271 aa, Rhizopus oryzae
SASDGGKVVAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVVQEQLTAHPTYKVIVTGHSLGGAQALLAGMDLYQREPRLSPKNLSIFTVGGPRVGNPTFAYYVESTGIPFQRTVHKRDIVPHVPPQSFGFLHPGVESWIKSGTSNVQICTSEIETKDCSNSIVPFTSILDHLSYFDINEGSCL

SEQ ID NO: 7 - protein, 267 aa, Aspergillus niger
TAGHALAASTQGISEDLYSRLVEMATISQAAYADLCNIPSTIIKGEKIYNSQTDINGWILRDDSSKEIITVFRGTGSDTNLQLDTNYTLTPFDTLPQCNGCEVHGGYYIGWVSVQDQVESLVKQQVSQYPDYALTVTGHSLGASLAALTAAQLSATYDNIRLYTFGEPRSGNQAFASYMNDAFQASSPDTTQYFRVTHANDGIPNLPPVEQGYAHGGVEYWSVDPYSAQNTFVCTGDEVQCCEAQGGQGVNNAHTTYFGMTSGACTW

SEQ ID NO: 8 - protein, 266 aa, Aspergillus tubingensis
TAGHALAASTQGISEDLYSRLVEMATISQAAYADLCNIPSTIIKGEKIYNSQTDINGWILRDDSSKEIITVFRGTGSDTNLQLDTNYTLTPFDTLPQCNSCEVHGGYYIGWISVQDQVESLVQQQVSQFPDYALTVTGHSLGASLAALTAAQLSATYDNIRLYTFGEPRSNQAFASYMNDAFQASSPDTTQYFRVTHANDGIPNLPPADEGYAHGVVEYWSVDPYSAQNTFVCTGDEVQCCEAQGGQGVNNAHTTYFGMTSGHCTW

SEQ ID NO: 9 - protein, 276 aa, Fusarium oxysporum
AVGVTTTDFSNFKFYIQHGAAAYCNSEAAAGSKITCSNNGCPTVQGNGATIVTSFVGSKTGIGGYVATDSARKEIVVSFRGSINIRNWLTNLDFGQEDCSLVSGCGVHSGFQRAWNEISSQATAAVASARKANPSFNVISTGHSLGGAVAVLAAANLRVGGTPVDIYTYGSPRVGNAQLSAFVSNQAGGEYRVTHADDPVPRLPPLIFGYRHTTPEFWLSGGGGDKVDYTISDVKVCEGAANLGCNGGTLGLDIAAHLHYFQATDACNAGGFSWRR

SEQ ID NO: 10 - protein, 273 aa, Fusarium heterosporum
TVTTQDLSNFRFYLQHADAAYCNFNTAVGKPVHCSAGNCPDIEKDAAIVVGSVVGTKTGIGAYVATDNARKEIVVSVRGSINVRNWITNFNFGQKTCDLVAGCGVHTGFLDAWEEVAANVKAAVSAAKTANPTFKFVVTGHSLGGAVATIAAAYLRKDGFPFDLYTYGSPRVGNDFFANFVTQQTGAEYRVTHGDDPVPRLPPIVFGYRHTSPEYWLNGGPLDKDYTVTEIKVCEGIANVMCNGGTIGLDILAHITYFQSMATCAPIAIPWKR

SEQ ID NO: 11 - protein, 278 aa, Aspergillus oryzae
DIPTTQLEDFKFWVQYAAATYCPNNYVAKDGEKLNCSVGNCPDVEAAGSTVKLSFSDDTITDTAGFVAVDNTNKAIVVAFRGSYSIRNWVTDATFPQTDPGLCDGCKAELGFWTAWKVVRDRIIKTLDELKPEHSDYKIVVVGHSLGAAIASLAAADLRTKNYDAILYAYAAPRVANKPLAEFITNQGNNYRFTHNDDPVPKLPLLTMGYVHISPEYYITAPDNTTVTDNQVTVLDGYVNFKGNTGTSGGLPDLLAFHSHVWYFIHADACKGPGLPLR

SEQ ID NO: 12 - protein, 278 aa, Penicillium camemberti
DVSTSELDQFEFWVQYAAASYYEADYTAQVGDKLSCSKGNCPEVEATGATVSYDFSDSTITDTAGYIAVDHTNSAVVLAFRGSYSVRNWVADATFVHTNPGLCDGCLAELGFWSSWKLVRDDIIKELKEVVAQNPNYELVVVGHSLGAAVATLAATDLRGKGYPSAKLYAYASPRVGNAALAKYITAQGNNFRFTHTNDPVPKLPLLSMGYVHVSPEYWITSPNNATVSTSDIKVIDGDVSFDGNTGTGLPLLTDFEAHIWYFVQVDAGKGPGLPFKR

SEQ ID NO: 13 - protein, 270 aa, Aspergillus foetidus
SVSTSTLDELQLFAQWSAAAYCSNNIDSKDSNLTCTANACPSVEEASTTMLLEFDLTNDFGGTAGFLAADNTNKRLVVAFRGSSTIENWIANLDFILEDNDDLCTGCKVHTGFWKAWESAADELTSKIKSAMSTYSGYTLYFTGHSLGGALATLGATVLRNDGYSVELYTYGCPRIGNYALAEHITSQGSGANFRVTHLNDIVPRVPPMDFGFSQPSPEYWITSGNGASVTASDIEVIEGINSTAGNAGEATVSVLAHLWYFFAISECLL

SEQ ID NO: 14 - protein, 270 aa, Aspergillus niger
SVSTSTLDELQLFSQWSAAAYCSNNIDSDDSNVTCTADACPSVEEASTKMLLEFDLTNNFGGTAGFLAADNTNKRLVVAFRGSSTIKNWIADLDFILQDNDDLCTGCKVHTGFWKAWEAAADNLTSKIKSAMSTYSGYTLYFTGHSLGGALATLGATVLRNDGYSVELYTYGCPRVGNYALAEHITSQGSGANFPVTHLNDIVPRVPPMDFGFSQPSPEYWITSGTGASVTASDIELIEGINSTAGNAGEATVDVLAHLWYFFAISECLL

SEQ ID NO: 15 - protein, 269 aa, Aspergillus oryzae
DVSSSLLNNLDLFAQYSAAAYCDENLNSTGTKLTCSVGNCPLVEAASTQSLDEFNESSSYGNPAGYLAADETNKLLVLSFRGSADLANWVANLNFGLEDASDLCSGCEVHSGFWKAWSEIADTITSKVESALSDHSDYSLVLTGHSYGAALAALAATALRNSGHSVELYNYGQPRLGNEALATYITDQNKGGNYRVTHTNDIVPKLPPTLLGYHHFSPEYYISSADEATVTTTDVTEVTGIDATGGNDGTDGTSIDAHRWYFIYISECS

SEQ ID NO: 16 - protein, 251 aa, Landerina penisapora
PQDAYTASHADLVKYATYAGLAYQTTDAWPASRTVPKDTTLISSFDHTLKGSSGYIAFNEPCKEIIVAYRGTDSLIDWLTNLNFDKTAWPANISNSLVHEGFLNAYLVSMQQVQEAVDSLLAKCPDATISFTGHSLGGALACISMVDTAQRHRGIKMQMFTYGQPRTGNQAFAEYVENLGHPVFRVVYRHDIVPRMPPMDLGFQHHGQEVWYEGDENIKFCKGEGENLTCELGVPFSELNAKDHSEYPGMH